(19) United States
(12) Patent Application Publication
(10) Pub. No.: US 2023/0086152 A1
(43) Pub. Date: Mar. 23, 2023

(54) USE OF DESLORATADINE AND SALTS THEREOF IN PREPARING DRUG FOR TREATING NEURODEGENERATIVE DISEASE RELATED TO MOTOR DYSFUNCTION

(71) Applicant: NANJING UNIVERSITY OF CHINESE MEDICINE, Nanjing (CN)

(72) Inventors: Xu SHEN, Nanjing (CN); Jiaying WANG, Nanjing (CN); Yujie HUANG, Nanjing (CN); Jian LU, Nanjing (CN)

(21) Appl. No.: 17/998,792

(22) PCT Filed: Dec. 14, 2021
(86) PCT No.: PCT/CN2021/137787

§ 371 Date: Nov. 14, 2022

(30) Foreign Application Priority Data
Aug. 17, 2021 (CN) 202110942414.6

(51) Int. Cl. A61K 31/4465; A61P 25/28

(52) U.S. Cl. CPC A61K 31/4465; A61P 25/28

(57) ABSTRACT

The present disclosure discloses a new use of desloratadine (DLT) and pharmaceutically acceptable salts thereof in preparing a drug for treating a neurodegenerative disease related to motor dysfunction. The present disclosure shows, through a large number of experimental tests, that desloratadine and the pharmaceutically acceptable salts thereof can improve the state of motor dysfunction in an SOD1-G93A model mouse in the behavioral experimental tests of Rotarod experimental test, gait monitoring experimental test and cage experimental test, and that desloratadine and the pharmaceutically acceptable salts thereof can effectively delay the onset time and prolong the survival cycle of the SOD1-G93A model mouse, and therefore, desloratadine and the pharmaceutically acceptable salts thereof can be used to treat neurodegenerative diseases related to motor dysfunction, including amyotrophic lateral sclerosis.

### TECHNICAL FIELD

The present disclosure relates to the field of drug therapy, in particular to a use of desloratadine and a pharmaceutical composition thereof in preparing a drug for treating a neurodegenerative disease related to motor dysfunction, and more specifically, the present disclosure relates to desloratadine and the pharmaceutically acceptable salts thereof, or the pharmaceutical composition thereof, which are capable of improving the state of motor dysfunction in an SOD1-G93A model mouse, and are used in the preparation of drugs for treating neurodegenerative diseases such as amyotrophic lateral sclerosis.

### BACKGROUND

Amyotrophic latera sclerosis (ALS) is a fatal neurodegenerative disease that is also known as “ALS”, which mainly affects the spinal cord, cerebral cortex and brainstem motor neurons. ALS usually occurs among middle-aged and elderly people or later. Patients will show progressive muscle atrophy and weakness after onset, and eventually die of respiratory failures under normal circumstances. Most patients survive only 2 to 4 years after onset. There are about 450,000 ALS patients in the world, and the prevalence rate is about 1/20,000. In 2018, China included ALS in the “First Batch of Rare Diseases List”. In 1993, it was found that SOD1 gene mutation is the causative gene of ALS, based on this, the first ALS model mouse, namely, SOD1-G93A model mouse came out in 1994, and this animal model has been widely used in ALS research. Although ALS is a rare disease, due to its extremely low cure rate and no specific available treatment drugs, it has brought great pain to ALS patients and their families. At present, there are only two clinical drugs for the treatment of ALS. Among them, riluzole was approved for marketing as early as 1995, but only recently has it been approved by the FDA as a treatment for ALS. Another drug of edaravone, was approved by the FDA in 2017 for the treatment of ALS. However, these two drugs could not cure ALS but can only temporarily prolong the survival of patients. Therefore, it is of great social value and practical significance to explore the pathogenic mechanism of ALS, search for new targets for the treatment of the disease, and develop effective drugs for the treatment of ALS.

The difficulty in the development of anti-ALS drugs is related to the unclear pathogenic mechanism. Current studies suggest that motor neuron damage is caused by the interaction of multiple pathogenic factors, including genetic factors, protein aggregation, neuroinflammation, oxidative stress, impairment of mitochondrial function, glutamate excitotoxicity and endoplasmic reticulum stress, and the like. (Kiernan M C, Vucic S, Cheah B C, et al. Amyotrophic lateral sclerosis [J]. Lancet, 2011, 377(9769): 942-955), and therefore, the present disclosure seeks for drugs to treat ALS from multiple possible pathogenic mechanisms of ALS.

Desloratadine is the main metabolite of loratadine, which is clinically used for the treatment of seasonal allergic rhinitis and chronic idiopathic urticaria. At present, there is no report that the desloratadine can improve the motor dysfunction of SOD1-G93A model mice, thereby playing a role in resisting amyotrophic lateral sclerosis. The present disclosure further finds that desloratadine has the effect of improving the motor dysfunction of SOD1-G93A model mice, thus the present disclosure further finds its potential for the treatment of new indications.

### SUMMARY

One objective of the present disclosure is to provide a new medicinal use of desloratadine and its pharmaceutically acceptable salts, that is, its use in preparing a drug for treating amyotrophic lateral sclerosis.

In the use, desloratadine and pharmaceutically acceptable salts thereof can be used as small molecule compounds to improve the motor dysfunction in an SOD1-G93A model mouse.

Another objective of the present disclosure is to provide the use of a pharmaceutical composition including desloratadine or a pharmaceutically acceptable salt thereof in preparing a drug for treating the amyotrophic lateral sclerosis, the pharmaceutical composition comprising desloratadine or its pharmaceutically acceptable salt and various pharmaceutically acceptable excipients.

The present disclosure further discloses the use of the pharmaceutical composition in preparing a drug for neurodegenerative diseases related to motor dysfunction.

The present disclosure discovers the new action mechanism and drug effect of desloratadine, and has the effect of improving the motor dysfunction of the SOD1-G93A model mouse. The compound can be used for the treatment of diseases related to amyotrophic lateral sclerosis and the motor dysfunction.

### BRIEF DESCRIPTION OF THE DRAWINGS

FIG. 1 illustrates that desloratadine can significantly improve the motor dysfunction of SOD1-G93A model mice in the Rotarod experimental test. * represents p<0.05, *** represents p<0.001, and ### represents p<0.001. One-way analysis of variance.

FIG. 2 illustrates that desloratadine can significantly improve the abnormal gaits of SOD1-G93A model mice in the gait monitoring experimental test. ** represents p<0.01, *** represents p<0.001, # represents p<0.05, and ### represents p<0.001. One-way analysis of variance.

FIG. 3 illustrates that desloratadine can significantly improve the declines on of limb grip strengths in SOD1-G93A model mice in the cage experimental test. ** represents p<0.01. *** represents P<0.001. One-way analysis of variance.

FIG. 4 illustrates that desloratadine can significantly delay the onset time of SOD1-G93A model mice. * represents p<0.05. Two-way analysis of variance.

FIG. 5 illustrates that desloratadine can significantly prolong the survival cycle of SOD1-G93A model mice. *represents p<0.05. Two-way analysis of variance.

### DETAILED DESCRIPTION OF THE EMBODIMENTS

The present disclosure will be further described below in combination with the specific embodiments, however, these embodiments should not be construed as limiting the present disclosure.

The implementation of experimental tests: Desloratadine improves the state of motor dysfunction in SOD1-G93A mice in the behavioral experimental tests of the Rotarod experimental test, gait monitoring experimental test and cage experimental test, effectively delays the onset time of SOD1-G93A model mice, and prolongs their survival cycle.

The present disclosure detects the effect of desloratadine on the improvement of the motor dysfunction state of mice in SOD1-G93A model mice. Experimental tests show that desloratadine can significantly improve the state of motor dysfunction in the behavioral experimental tests of the SOD1-G93A model mice in the Rotarod experimental test, the gait monitoring experimental test and the cage experimental test.

1. The Principle of Experimental Tests

1) Rotarod experimental test: SOD1-G93A model mice will have motor dysfunction when they are onset, and therefore, the motor coordination and balance function of the forelimb and hind limbs can be assessed by detecting the duration that the mice stay on the rotarod.

2) Gait monitoring experimental test: SOD1-G93A model mice show unstable gaits and resting tremor at the onset of the disease, gradually develop into hindlimb atrophy, and develop into complete paralysis of both hindlimbs in the late stage of the disease with the significantly shortened gait length, The motor coordination function at the front and rear limbs of the mice is evaluated through the gait analysis.

3) Cage experimental test: The SOD1-G93A model mice show atrophy of the hind limbs and decreased grip strength of the limbs after the onset of the disease. The handing strength at the limbs of the mice is detected by the cage experimental test.

4) Detection on onset time and survival cycle: The duration from onset to death of SOD1-G93A model mice is shorter, which is similar to the human ALS. Whether it can effectively delay the onset of the disease and prolong the survival cycle is an important indicator.

2. Materials and Methods of Experimental Test

1) Animal grouping: Experimental test mice B6SJL-BTg (SOD1-G93A) 1Gur/J (002726) are purchased from Jackson Laboratory in the United States. A total of 48 SOD1-G93A mice are obtained by the gene identification, and their littermates are used as negative control, and they are randomly divided into 4 groups, which are: \textcircled{1} a wild-type solvent group \textcircled{2} a wild-type DLT group (20 mg/kg/day), \textcircled{3} a model solvent group and \textcircled{4} a model DLT group (20 mg/kg/day). All mice are administered from the age of 56 days (8 weeks old), and 12 mice in each group are sacrificed from the beginning of administration to the age of 120 days. Histopathological, biochemical and immunological analyses are performed on tissues such as blood, muscles and spinal cords from eyeballs of mice. Compounds are administered to the remaining mice until their deaths, which is used to detect the onset time and survival cycle of mice.

2) Rotarod experimental test: From the age of 60 days, the motor function of SOD1-G93A model mice is detected by the rotarod apparatus. Mice are trained for one week to become familiar with the rotarod apparatus. The mice are tested twice a week from the age of 70 days. At the beginning of the experimental test, the mice are individually placed on the rotating cylinder of the rotarod apparatus and rotated at a constant speed of 12 revolutions per minute. Motor coordination and balance are evaluated for each mouse by measuring the mouse's total movement time on the rotarod apparatus. Three trials are performed for each mouse, and the longest dwell time before falling is recorded, and the longest dwell time is set at 180 seconds.

3) Gait monitoring experimental test: In order to obtain footprints, the forefeet and hind feet of SOD1-G93A model mice are painted with red and green non-toxic pigments, respectively. The mice walk along a track with a length of 50 cm and a width of 10 cm, and the gaits of the mice are recorded. All of the mice are tested weekly with three runs each time. Footprints of the mice are analyzed, and the average of three result statistics is used in the analysis for recording the gait lengths of the mice.

4) Cage experimental test: From the 8th week, the test is carried out once a week. During the experimental test, a soft pad is placed on the ground to protect the mice, and then the mice are placed on the cage cover. After confirming that the mice have held the cage cover, the cage cover is flipped quickly and the timing is started at the same time. The maximum time for the mice to grip the cage cover is recorded, the maximum time is set to be 90 seconds, and the specific time is recorded when it is less than 90 seconds. Each experimental test is repeated three times and the best score is taken as the record.

5) Detection of onset time and survival cycle: The onset time of transgenic mice is defined as the dates when they first fall from the rotarod apparatus and will not persist for 180 seconds. The dates of deaths of the transgenic mice are defined as the deaths of the mice if the righting reflex cannot be completed within 30 seconds when the mice are lying on their sides, and the days are recorded as the death dates of the mice.

3. The Results of Experimental Tests

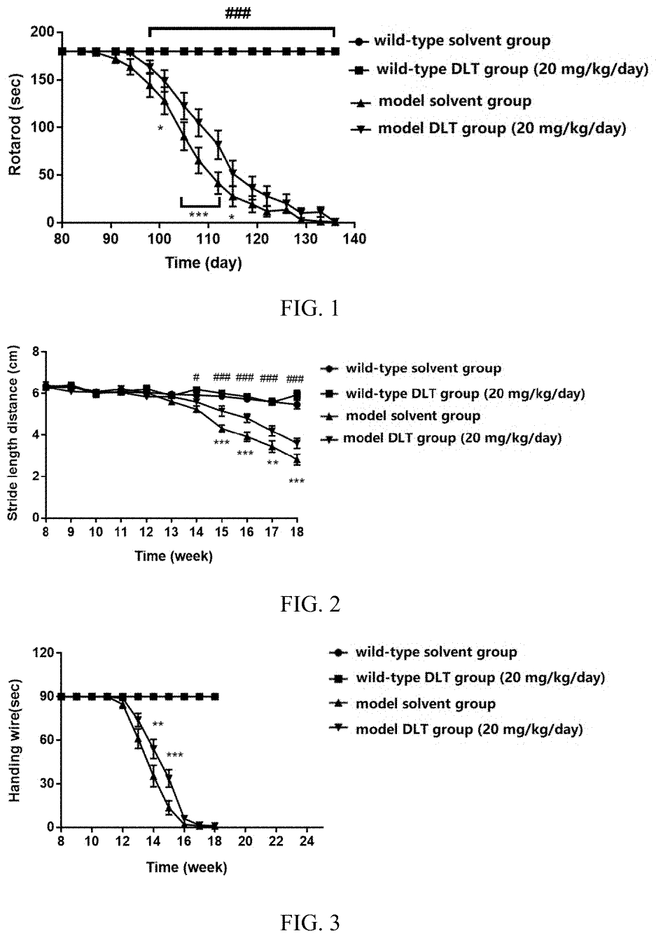

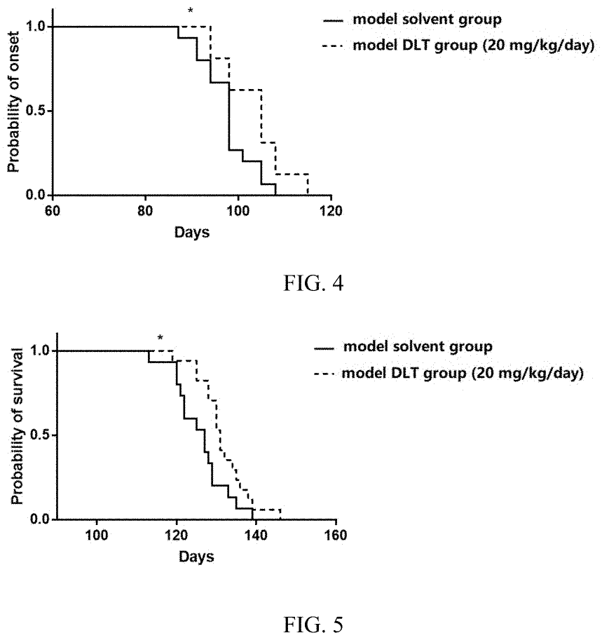

The results are as illustrated in FIG. 1. Compared with the model solvent group, the mice in the model DLT group perform better in the Rotarod experimental test, and the mice in the model DLT group stayed on the rotarod for a much longer period of time (days 101 to 115) than the mice in the model solvent group, which shows that desloratadine can improve the motor coordination and balance function of the mice. As illustrated in FIG. 2, the gait length analysis results of the mice in the model DLT group after the 15th week are significantly better than the mice in the model solvent group, which shows that desloratadine can improve the abnormal gaits of the model mice. As illustrated in FIG. 3, the mice in the model DLT group at the age of 14 to 15 weeks persist better than the mice in the model solvent group in the cage experimental test, which shows that desloratadine can enhance the muscle strengths of mice. As illustrated in FIG. 4, the administration of desloratadine can delay the time when the SOD1-G93A model mice fall from the rotarod for the first time, and delay the onset time (the model solvent group=97.6±1.473 d, the model DLT group=103.4±1.705 d, p<0.05). As illustrated in FIG. 5, the time delays on falling to complete the righting reflex of the mice in the model DLT group could indicate that desloratadine has the effect of prolonging the survival cycle of SOD1-G93A model mice (the model solvent group=126±1.721 d, the model DLT group=131.6±1.51 d, p<0.05).

The above-mentioned embodiments only express a few preferred implementations of the present disclosure. It should be pointed out that for those of ordinary skilled in the art, without departing from the concept of the present disclosure, a plurality of modifications and improvements can be made, which shall also be regarded as the protection scope of the present disclosure.

1-5. (canceled)

6. A use of desloratadine and pharmaceutically acceptable salts thereof in preparing a drug for treating a neurodegenerative disease related to motor dysfunction, wherein the desloratadine and the pharmaceutically acceptable salts thereof are capable of improving motor dysfunction of an SOD1-G93A model mouse.

7. A use of desloratadine and pharmaceutically acceptable salts thereof in preparing a drug for treating amyotrophic lateral sclerosis.

8. A use of a pharmaceutical composition in preparing a drug for treating amyotrophic lateral sclerosis, wherein the pharmaceutical composition comprises desloratadine or pharmaceutically acceptable salts thereof, and a pharmaceutically acceptable auxiliary material.
